(19) United States
(12) Patent Application Publication
(10) Pub. No.: US 2017/0233758 A1
(43) Pub. Date: Aug. 17, 2017

(54) HERBICIDE TOLERANT TRIPLE GENE INSECTICIDAL COTTON AND OTHER PLANTS

(71) Applicants: Daniyal Jawed Qureshi, Lahore (PK); Hamza Nadeem Qureshi, Lahore (PK)

(72) Inventors: Daniyal Jawed Qureshi, Lahore (PK); Hamza Nadeem Qureshi, Lahore (PK)

(21) Appl. No.: 15/162,632

(22) Filed: May 24, 2016

(30) Foreign Application Priority Data
Feb. 15, 2016 (PK) 96/2016

(51) Int. Cl. C12N 15/82; C12Q 1/68; C07K 14/325; G01N 33/68

(52) U.S. Cl. CPC C12N 15/8286; C12N 15/8274; G01N 33/6854; C12Q 1/6806; C07K 14/325

(57) ABSTRACT

The present invention refers to the next-generation three genes cotton (Gossypium hirsutum) expressing chloroplast-targeted herbicide tolerant gene resistant to broad and narrow-leaved weedicide sprays and two B. thuringiensis insecticidal genes δ-endotoxin Cry2A and vegetative insecticidal protein gene VIP3A. Both Cry2A and VIP3A genes have different modes of action in controlling a wide spectrum of lepidopteron insect pests, therefore, likely risk of pest resistance will be minimized which is a prevalent problem with single gene Bt cotton expressing Cry1Ac in Pakistan. The invention comprises novel nucleic acid segments encoding proteins comprising herbicide tolerance, Cry2A and VIP3A insecticidal toxins. The polynucleotide segments are revealed, as are Agro-bacterium-mediated transformation vectors holding the nucleic acid segments, plants transformed with claimed segments, methods for transforming plants, and methods for controlling plant infestation by pests.

The following specification particularly describes the invention and the manner in which it is to be performed

### FIELD OF INVENTION

The present invention relates to genetic engineering of plants and in specific to herbicide tolerant as well as insect resistant transgenic cotton plant. More particularly, several embodiments are related to novel polynucleotide segments. It also recounts to methods of enhanced chloroplast-targeted expression of EPSP syntheses protein as well as two insecticidal proteins in transgenic plant transformed with transgenes, resulting in effective control of broad and narrow-leaved weeds as well as susceptible target pests.

### BACKGROUND OF THE INVENTION

Cotton is an important source of raw material to the textile industry and has a share of 1.4% in GDP and 6.7% in Pakistani agriculture. During Jul.-Mar. 2013-14, textile industry earned foreign exchange of USS 10.385 billion. The cotton crop was cultivated on an area of 2806 thousand hectares during 2013-14, 2.5% less than area of 2879 thousand hectares in 2012-13.

The production stood at 12.8 million bales during the period 2013-14 against the target of 14.1 million bales, showing decline of 9.2 percent against the target and decline of 2.0 percent over the last year production of 13.0 million bales (Economic Survey of Pakistan, 2014).

Insect pests are a major problem for cotton crop in cotton areas around the world.

Cotton in Pakistan is mainly damaged by a number of lepidopteron larvae like spotted bollworms, American bollworms, pink bollworms and Armyworms, etc. These larvae feed on cotton bolls or flowers, and therefore cause direct and great yield losses. Public and private sector institutions have made seeds of 16 Bt cotton varieties provisionally approved by the Punjab Seed Council (PSC) between 2010 and 2012 in the three intensive cotton growing provinces of Punjab, Sindh and Baluchistan. Of the 16 approved Bt cotton varieties (including one Bt cotton hybrid), 15 contain the crylAc gene (MON531 event), while the Bt cotton hybrid GFM-2085 expresses the fusion gene cry1Ac and cry1Ab (James, 2013). Cotton production in Pakistan has remained stagnant due to many reasons. Virus and pest attack on cotton has significantly hindered cotton production. Although the country has adopted transgenic cotton (single-gene Bt) over the area of 86%; even then this resulted in incidence of pink bollworm and other lepidoptrous pests during 2013-14 (Khuhro et al., 2015). Recently, in Sindh, pink bollworm (Pectinophora gossypiella) has become a real threat to conventional and Bt varieties of cotton. Its attack to fruiting bodies of cotton ranges from 20-30 percent (Ahmed, 2013). Despite availability of Bt varieties Bollguard-II (BG-2) and Roundup Ready Flex (RRF) cotton in the markets of Southern Punjab and Sindh, it was surprising that the farmers were reporting pest development in the cotton fields due to low levels of Bt toxins in GM cotton crop (GM Watch, 2014). In the light of above scenario, one can envisage the dire future with increasing field-evolved insect resistance against Bt toxins. The lower expression level of Bt toxin, instead of killing, is helping the lepidopteron larvae to develop resistance. Bravo et al. (2013) are also of the view that a major threat for the use of Cry toxins in transgenic plants is the appearance of insect resistance. Evolution of resistance to Bt-crops in the field has been documented for at least five different insect species (van Rensburg, 2007; Tabashnik et al., 2008; Bagla, 2010; Storer et al., 2010; Gassmann et al., 2011). To delay evolution of pest resistance to transgenic crops producing insecticidal proteins from Bacillus thuringiensis, is the “pyramid” strategy which uses plants that produce two or more toxins that kill the same pest (Wei et al., 2015; Brevault et al. 2013). Expanded use of transgenic crops for insect control will likely include more varieties with combinations of two or more Bt toxins, and novel Bt toxins such as vegetative insecticidal proteins (VIPs) (Tabashnik et al., 2009). Therefore, it has become quintessential to develop new GM cotton cultivars in Pakistan with stacked Bt genes, other than Cry1Ac, displaying elevated targeted-expression of insecticidal toxin. The present investigation aims at transforming cotton with multiple genes to have targeted expression in green tissues enabling cotton plant to combat effectively with the resistant insect pests.

### SUMMARY OF THE INVENTION

An objective was to develop innovative methods for the chloroplast-targeted expression of herbicide tolerant gene, a Bt crystal protein gene Cry2Ab and a vegetative insecticidal protein VIP3A in stacked-triple-gene transformed cotton plants. The enhanced synergetic expression of Cry2Ab and VIP3A makes this next generation transgenic cotton more tolerant to lepidopteran insects than first generation single gene Bt cotton.

The invention addresses another limitation of the prior art: development of insect resistance. Particularly, the present invention offers a superior strategy for the postponement or elimination of the development of insect resistance to Cry1A δ-endotoxins, Bt proteins most commonly expressed by transgenic cotton in Pakistan. The revealed methods involve expression of Cry2A class of B. thuringiensis δ-endotoxins and VIP3A class of B. thuringiensis vegetative insecticidal toxins. Both Cry2A and VIP3A have no significant homology to each other and have different modes of action in the midgut of insect (Lee et al., 2003), and as such are expected to control insects that have become resistant to Cry1A δ-endotoxins (Tabashnik et al., 2009).

In first construct cassette, the present invention provides a G. hirsutum codon optimized purified DNA construct comprising synthetic EPSP synthase protein-encoding region localized to a chloroplast, or localized to a plant cell nuclear genome and is operably linked to a region encoding a chloroplast transit peptide (cTP), which is one means of enabling localization of EPSPS protein to chloroplast. In certain embodiments, the EPSPS gene comprises the sequence of SEQ ID NO: 17

In the second construct cassette, the instant invention provides cotton optimized DNA construct comprising B. thuringiensis VIP3A vegetative insecticidal protein-encoding region localized to chloroplast, or localized to a plant cell nuclear genome and is operably linked to a region encoding a chloroplast transit peptide (cTP), which is one means of enabling localization of VIP3A protein to chloroplast. In certain embodiments, the VIP3A gene comprises the sequence of SEQ ID NO: 18.

In the third construct cassette, G. hirsutum optimized DNA construct comprising Cry2A B. thuringiensis δ-endotoxin-encoding region localized to plastid or chloroplast, or localized to a plant cell nuclear genome and is operably linked to a region encoding a chloroplast transit peptide (cTP) under green tissue-specific promoter, which is one means of enabling localization of Cry2A protein to chloroplast. In certain embodiments, the Cry2A gene comprises the sequence of SEQ ID NO: 16.

In one vector construct, the Cry2A endotoxin gene is expressed under constitutive promoter CamV35S attached to a Chloroplast Transit Peptide as localization signal peptide.

There exists, in the Pakistani agriculture, a necessity to raise a cotton plant that shows multi-tolerant characteristics i.e. weedicide tolerance and better insect resistance so that yield losses due to weeds as well as damage to cotton crop by insect pests may be reduced. The present invention insect resistant triple-gene cotton plant would reduce the need to apply chemical pesticides that might be harmful to other beneficial insects and the environment. Moreover, two insecticidal genes having differing mode of action would help in delaying the insect resistance to Bt genes, which is prevailing problem in Pakistani agriculture with single Bt gene cotton.

Furthermore, herbicide resistance trait in cotton plant would abolish the labor intensive manual tillage for weed removal and thus, enabling farmers to get rid of unwanted herbs by one or two sprays of non-selective herbicide. In this way, farmers would save both labor cost and time and would glean better yield from field.

The present invention, therefore, relates to herbicide and insect resistant next generation triple-gene transgenic cotton. It also relates to methods of identifying plant materials derived there from. Genetically modified herbicidal and insecticidal cotton in the framework of this application denotes the innovative triple-gene transgenic cotton plant described herein.

In addition, the invention also includes a method for the enhanced expression of EPSPS enzyme conferring herbicide tolerance in crop plants, and Cry2A and VIP3A insecticidal protein genes by expressing in chloroplasts (green tissues) of the cotton plant.

### BRIEF DESCRIPTION OF DRAWINGS

FIG. 1 is a graph illustrates various temperatures at which PCR amplification is carried out.

### DETAILED DESCRIPTION OF THE INVENTION

Methods of Removing Weeds from Crop Field

Crops are forced to compete for sunlight and nutrients because of unwarranted weed growth that often leads to substantial yield losses. Conventionally, farmers control weeds in their farms by tandem technique of soil-tilling or by herbicide application. Soil tilling is effective but is cumbersome and needs intensive labor, time and money. On the other hand, herbicides cannot discriminate between crop plants and weed plants, so conservative agricultural systems can use ‘selective’ herbicides only. Such herbicides do not damage the crop, but are not effective at removing all types of weeds. If herbicide resistant crops are used, farmers can remove all weeds in a single, swift application of ‘non-selective’ herbicides. This is cost, labor and time saving because it needs less spraying, less labor, less traffic on the field, and lower operating costs. Glyphosate is a ‘non-selective’ broad-spectrum herbicide that restricts EPSP synthase which is an essential enzyme in the aromatic amino acids and vitamin synthesis pathway. Once this EPSPS enzyme is blocked, the plant is deprived of food and is eventually killed. Glyphosate is useful for weed control and has no significant direct impact on animal life, and is not persistent. It is highly effective and among the safest of agrochemicals to use. Unfortunately, it is equally effective against crop plants. There are several means for the modification of crops to be tolerant to glyphosate. One approach is to genetically engineer crop plant with a soil bacterium gene that yields a glyphosate-tolerant form of EPSPS. The implementation of glyphosate-based crop production system is one of the most significant revolutions in the history of agriculture.

### Methods of Controlling Insect Infestation in Crops

Bacillus thuringiensis is a Gram-positive soil bacterium that is well known for its production of proteinaccous parasporal crystals (Cry proteins) and vegetative insecticidal proteins (VIPs), which are toxic to a variety of Lepidopteran, Coleopteran, and Dipteran insect larvae.

Crystal proteins are produced by B. thuringiensis during sporulation phase which are primarily toxic to certain crop infesting species of insects. Various compositions comprising Bt strains producing Crystal toxic proteins with insecticidal activity have been used commercially as environmentally-acceptable topical insecticides because of their toxicity to the specific target insect, and non-toxicity to plants and other beneficial non-target organisms. Insect larvae ingest these δ-endotoxin crystals which after solubilization in the midgut of the insect release protoxin form of the δ-endotoxin that is processed subsequently to an active toxin by midgut protease. The activated toxins recognize and bind to the brush-border of the insect midgut epithelium through receptor proteins.

Molecular genetic techniques have enabled molecular biologists to recognize and isolate various δ-endotoxin genes and determine their DNA sequences. These genes were further truncated for effective use in higher crop plants and were used to develop genetically engineered Bt products that have been approved for commercial use.

On the other hand, in recent years, a number of insecticidal proteins expressed during the vegetative growth phase of Bacillus thuringiensis have been characterized (Yu et al., 2011; Palma et al., 2012). These vegetative insecticidal proteins (VIPs) contrast with the widely investigated B. thuringiensis δ-endotoxins, or insecticidal crystal proteins, which form parasporal inclusions primarily during the sporulation phase (Schnepf et al., 1998). One of the interesting features of the Vip3A protein is that it shares no sequence homology with the known δ-endotoxins. The VIPs have shown a broad insecticidal spectrum, including activity toward a wide variety of lepidopteran and also coleopteran pests (Gulzar and Wright, 2015; Lee et al., 2003). The action of Vip3A has been examined and shown to be at the level of insect midgut epithelium, where binding to midgut cells is followed by progressive degeneration of the epithelial layer (Lee et al., 2003).

### Chloroplast Targeting

In the present invention, expression vectors were designed to precisely target the expression of polypeptides into the chloroplast of the transformed plant using sequences encoding chloroplast transit peptide (cTP) signals. These signals tagged at N-terminal of the coding sequence of the gene of interest help in the chloroplast-targeted enhanced expression of transgenes after cellular processing, such as, transcription, translation and expression of the encoded protein. The frequency of recovery of morphologically and phenotypically normal plants is also increased by using cTP signal peptides. To offer supplementary shield against likely development of insect resistance via a high dose strategy (Gryspeirt and Gregoire, 2012; Tabashnik et al., 2013), increased expression of insecticidal transgenes is particularly valuable. Enhanced expression of transgenes is also desirable as it provides sustained insect protection in occurrences where insecticidal gene expression decreases due to environmental conditions.

Chloroplast-targeted and non-targeted (cytosolic) expression of herbicide tolerant (HT) gene, Cry2Ab and VIP3A showed significant boost in levels of HT, Cry2Ab and VIP3A chloroplastlocalized proteins in plants transformed with the chloroplast-targeted signals relative to plants transformed without signal peptides. This result was in accordance with the published reports (Kiani et al., 2013)

Chloroplast targeting transit peptides, as revealed in the present invention, were found equally useful for the targeted expression of glyphosate resistant gene as well as two insecticidal genes. In this invention, plants transformed to express a protein conferring glyphosate resistance are transformed with a cTP that targets the peptide to the cell's chloroplasts. Glyphosate inhibits the shikimate pathway which leads to the biosynthesis of aromatic compounds including amino acids and vitamins. Specifically, glyphosate inhibits the conversion of phosphoenolpyruvic acid and 3-phosphoshikimic acid to 5-enolpyruvyl-3-phosphoshikimic acid by inhibiting the enzyme 5-enolpyruvyl-3-phosphoshikimic acid synthase (EPSP synthase or EPSPS). Supplemental EPSPS, conferred via insertion of a transgene encoding this enzyme, allows the cell to resist the effects of the glyphosate. Thus, as the herbicide glyphosate functions to kill the cell by interrupting aromatic amino acid biosynthesis, particularly in the cell’ chloroplast, the cTP allows increased resistance to the herbicide by concentrating what glyphosate resistance enzyme the cell expresses in the chloroplast, i.e. in the target organelle of the cell. Plants transformed in the present invention, also accumulate insecticidal toxins of Cry2A and Vip3A at elevated levels in green tissues, such as leaves, which are the primary targets of the chewing insects. Therefore, these concentrated toxins are quite effective in controlling lepidopteran insect larvae.

Preferred cTPs of the present invention include those targeting transgenes to chloroplasts. Specific examples of preferred cTPs include the Petunia EPSPS protein cTP, Petunia Cab gene for Chlorophyll a/b binding protein cTP and Ricin Cab gene for Chlorophyll a/b binding protein cTP. These cTPs are illustrated by the polynucleotides shown in SEQ ID NO:13, SEQ ID NO:14, and SEQ ID NO:15. Polypeptide sequences resulting from these polynucleotides are shown in SEQ ID NO:20, SEQ ID NO:21, and SEQ ID NO:22. Also, included in the present embodiment is the 1.558 kb (SEQ ID NO:23) long green tissue-specific promoter and its allied untranslated region of the PNZIP (Ipomoea nil leucine zipper) gene from Ipomoea nil (Japanese morning glory) attached to N-terminal of Cry2A and/or VIP3A•gene.

### Use of Introns in Expression Vectors

An intron may also be included in the DNA expression construct for optimized expression in monocotyledonous and dicotyledonous plants (Morello and Breviario, 2008). Such an intron is classically inserted towards the 5′-end of the sequence of mRNA. The sequence of intron may possibly be obtained from first or second intronic regions of the G. hirsutum SCFP gene, Cab gene for Chlorophyll a/b binding protein gene of Brassica rapa, and Cab gene for Chlorophyll a/b binding protein gene of Petunia hybrida (Kiani, unpublished data). As exposed herein, these introns (SEQ ID NO:13, SEQ ID NO:14, SEQ ID NO: 15 and SEQ ID NO:23) are valuable in the present invention.

Identification and Isolation of Insecticidal B. thuringiensis δ-Endotoxins and Genes

The method pronounced in this invention is anticipated to be used to achieve significantly enhanced expression of Cry2A B. thuringiensis endotoxins and vegetative insecticidal protein VIP3A extracted as given below. Donovan et al. (1992) have described how to identify new B. thuringiensis strains encoding crystalline endotoxins with insecticidal activity. It is consisted of following steps: isolation of the Bt endotoxin, amino acid sequencing, back-translation, designing of oligonucleotide probe, followed by identification and cloning of the endotoxin gene by hybridization. Those skilled in the art are familiar with this process of identification of Cry and VIP genes.

Improved coding sequences of G. hirsutum-optimized Cry2A and VIP3A can be designed by using bioinformatics tools that use the newly available genome data of G. hirsutum and G. barbedense. This optimization helps in improved expression of transgenes of prokaryotic origin in eukaryotic plants. Enhanced levels of targeted expression of Cry2A, VIP3A and herbicide tolerant gene in transgenic cotton plants have been achieved through methods described in this invention.

### Construction of Plant Expression Vectors

Designing of plasmid vectors for tissue-specific targeting of gene expression in transgenic plants typically includes tissue-specific promoters and also may include other tissue specific control elements such as enhancer sequences. Promoters which direct specific or enhanced expression in certain plant tissues will be known to those of skill in the art in light of the present disclosure.

To accomplish chloroplast-specific expression functionally, it is also envisaged that it may be done by introducing a constitutively expressed gene attached to some suitable transit peptide flanked by gene at 3′ terminal and promoter towards 5′ terminal of transit peptide. For instance, when VIP3A gene coding for the vegetative insecticidal protein from B. thuringiensis is introduced, it is expressed in all tissues using the Cauliflower Mosaic Virus 35S promoter. But when a chloroplast transit peptide is attached to 5′ end of VIP3A, it gives normal expression in all tissues and higher expression in chloroplasts. Alternatively, an Ipomoea nil leucine zipper (PNZTP) gene promoter (SEQ ID NO: 23) may be used for targeted expression of VIP3A or Cry2A gene.

### Nucleic Acid Composition

In one imperative embodiment, the triple gene cotton exhibits a novel genotype comprising four expression cassettes, that is, three cassettes of transgenes and one cassette of selectable marker gene.

The first cassette comprises an appropriate promoter operably linked to a gene that encodes for an EPSP synthase protein enzyme, which renders transgenic cotton plant tolerant to non-selective herbicide spray.

A 108 nucleotides long chloroplast transit peptide (SEQ ID NO:9) is attached towards N-terminal, at the start of EPSPS gene for the directional expression of EPSPS gene inside green tissues.

An untranslated region (5′ UTR) comprising 141 nucleotides (SEQ ID NO: 9) flanks between promoter and cTP.

In the second cassette, VIP3A gene is tagged at N-terminal with 144 bp long chloroplast transit peptide (SEQ ID NO: 11) for targeted expression of vegetative insecticidal protein in chloroplasts of transgenic plant.

A 5′UTR of 99 nucleotides (SEQ ID NO: 11) is inserted between cTP and promoter.

The third cassette comprises a promoter region attached to a 177 nucleotides long 5′ UTR (SEQ ID NO: 7), which is further joined with 216 bp chloroplast transit peptide (SEQ ID NO:7), for chloroplast-localized expression of Cry2A gene.

The fourth cassette comprises a marker gene, already incorporated into the plant expression plasmid vector, which when expressed can be used as a selectable marker. In one embodiment of the present invention, the selectable marker gene is either hygromycin or kanamycin.

All the transgenes (EPSPS, Cry2A and VIP3A), at their C-terminals, are linked separately to polyadenylation signals from nopaline synthase gene (NOS) of Agrobacterium tumefaciens, from proteinase inhibitor II gene.

The four dicotsettes may be inserted into the plant on the same or different plasmids.

If first, second, third and fourth cassettes are present on the same plasmid and introduced into the cotton genome via Agrobacterium-mediated transformation method, they may be present in the same or different T-DNA regions.

In one embodiment, all four cassettes are on the same T-DNA region.

In second embodiment, first, second and fourth cassettes are present on the same T-DNA region.

In third embodiment, first, third and fourth cassettes are on the same T-DNA region.

In fourth embodiment, first, second and fourth cassettes are on the same T-DNA region.

In fifth embodiment, all four cassettes are on the same T-DNA region, and Cry2A is expressed under green tissue-specific PNZIP promoter.

In one aspect of the present invention, there is provided a polynucleotide sequence as defined above consisting of the sequence of SEQ ID NO: 19. In an additional embodiment, the said plant is a cotton plant and that plant is herbicide tolerant and insect resistant cotton plant, which has been generated using Agrobacterium-mediated method of plant transformation.

### Cotton Transformation

Transformation and regeneration of cotton is now a well-established procedure, typically based on Agrobacterium tumefaciens mediated transfer of foreign DNA into cotton genome and regeneration of said plant parts into fully fertile transgenic cotton plants.

Agrobacterium-mediated transformation is a normally used method for transformation of dicotyledonous plants. The DNA of interest is cloned into a binary plasmid vector in between left and right T-border consensus sequences, called T-DNA region. This binary vector is transmitted into Agrobacterium cell through electroporation, which is subsequently used to infect plant tissue. The T-DNA region of the vector comprising the foreign DNA is integrated into the plant genome. The selectable marker gene cassette and trait gene cassette(s) may be present on the same or different T-DNA regions in the same vector, or different vectors. In one embodiment of the present invention, the gene cassettes are present on the same T-DNA region.

The next step after transformation is the selection and regeneration of transgenic plants via selection pressure of antibiotic drugs corresponding to appropriate marker gene (hygromycin or kanamycin), and progeny retaining the foreign DNA. The composition of suitable regeneration media is well known to any skilled man.

The progeny plants achieved thus, as described in the present invention, have herbicidal or insecticidal effects. These plants show tolerance to non-selective’ herbicide sprays and are resistant to insects from lepidopteran species comprising Heliothis sp; Helicoverpa sp., Pictinophora sp. and Spodoptera sp. Which may invade it. The term—Invade herein refers to infest, feeding or harm by one or more insect pests. Consequently, self-defense mechanism is exhibited by the transgenic cotton plants of the present invention against invasion by pest insects such as Armyworm (Spodoptera litura) and cotton bollworm (Helicoverpa zea). Thus, a fewer insecticide sprays are needed for the cultivation of invented plant in comparison to a wild-type cotton plant of the same cultivar and a minimal level of yield loss through insect pests has been observed.

The current invention is not restricted to the aforementioned transgenic cotton plant only, but is further stretched to take account of any plant material acquired from it, including seeds if at least one of the current inventive polynucleotides is contained by them.

The present invention covers plants which are obtained from a cross-breeding with the current transgenic cotton plant or a resultant there from by orthodox breeding or other procedures.

The present invention is also extended to plant material achieved from the transgenic plant that may contain additional, changed or fewer polynucleotide sequences matched with the transgenic cotton plant or display other phenotypic features. For instance, if someone desires to generate a new event by transforming the plant material derived from the current transgenic cotton plant which displays an additional feature, such as a third insect resistance gene or herbicidal gene—a process known as gene stacking.

The present invention further provides a method to achieve chloroplast-targeted higher expression of herbicidal EPSPS tolerant gene as well as insecticidal Cry2A and VIP3A genes in dicotyledonous transgenic plants, without undesirably affecting the normal phenotype and agronomic characteristics of the transgenic plants.

The present invention also allows getting herbicidal as well as insecticidal toxins at levels up to 30 times higher than that exhibited by existing procedures.

The method. described herein enables transgenic plants expressing herbicide tolerant gene, δ-endotoxin Cry2A and/or vegetative insecticidal VIP3A gene to be used as an alternative to plants expressing first generation Cry1A-type B. thuringiensis δ-endotoxins. These next generation toxins Cry2A and VIP3A with their coupled-effect will be used both for control as well as resistance management of significant insect pests, including Heliothis sp., Helicoverpa sp., Pictinophora sp. and Spodoptera sp. It is also anticipated that two insecticidal toxins having different mode of action in insect midgut will increase the effectiveness against target insect pests and will decrease the possibility of evolved resistance against these toxin proteins. The higher expression of these toxins in green tissues will further reduce the chances of insect resistance.

The method of expressing triple genes—herbicide tolerant gene, Cry2A and VIP3A—in green tissues of cotton plants includes the following steps:
i. Designing and constructing a polynucleotide consisting of a suitable promoter operably joined to a sequence encoding chloroplast transit peptide (cTP), which is further tagged to DNA sequence encoding either herbicide tolerant protein, or δ-endotoxin Cry2A, or vegetative insecticidal protein VIP3A, which is further linked at 3′ end to a suitable terminator sequence. The genes thus tagged with cTP will yield fusion proteins consisting of N-terminal chloroplast transit peptide and a polypeptide of either herbicide tolerant protein, or δ-endotoxin Cry2A, or vegetative insecticidal protein VIP3A.
ii. Transforming the plant with DNA construct of step (i) so that the transgenic plant expresses the fusion proteins.

The plant transformed by the method revealed herein may either be dicotyledonous plant or monocotyledonous plant.

Any variety of dicotyledonous plant including fiber, legume, tuber or fruit plant and any variety of species of monocotyledonous plant is encompassed by the present invention.

In preferred embodiments, the dicot is a cotton, tomato, and potato plant or cell. While in preferred embodiments, the monocot plant is maize, rice, wheat, and sugarcane plant.

### Laboratory Insect Bioassays of Transgenic Plant Events

To identify a transgenic plant expressing high levels of the herbicide tolerant protein, Cry2A δ-endotoxin and VTP3A vegetative insecticidal protein of interest, screening is necessary of the herbicide or antibiotic resistant transgenic, regenerated plants (TO generation) for herbicidal and insecticidal activity and/or expression of the genes of interest.

Various methods well known to those skilled in the art may help in accomplishment of this task, including but not limited to: (1) taking leaf samples from the transgenic TO plant and directly assaying the leaf for activity against susceptible insects in comparison with tissue obtained from a non-transgenic, negative control plant. For example, TO transgenic cotton plants expressing Cry2A δ-endotoxin and VIP3A vegetative insecticidal protein can be identified by assaying leaf tissue obtained from such plants for activity against Heliothis sp., Helicoverpa sp., Pictinophora sp. and Spodoptera sp.; (2) analysis of extracted protein samples by enzyme linked immuno sorbent assay (ELISA) specific for the gene of interest (herbicide tolerant protein, Cry2A and VIP3A); or (3) reverse transcriptase PCR™ (RT PCR™) to classify events of the expression of the genes of interest.

Method of Expressing Herbicide Tolerant Protein, cry2a δ-Endtoxin and vip3a Vegetative Insecticidal Proteins in Progeny Plant

The inventor of the present invention further anticipates that the method disclosed in this invention encompasses a method of generating a transgenic progeny plant. The method of generating such progeny embraces: the process of expressing herbicide tolerant protein, Cry2A δ-endotoxin and VIP3A insecticidal toxin in a plant disclosed herein includes steps of: (i) designing and constructing a polynucleotide consisting of a suitable promoter operably joined to a sequence encoding chloroplast transit peptide (cTP), which is further tagged to DNA sequence encoding either herbicide tolerant protein, or 6-endotoxin Cry2A, or vegetative insecticidal protein VIP3A, which is further linked at 3′ end to a suitable terminator sequence. The genes thus tagged with cTP will yield fusion proteins consisting of N-terminal chloroplast transit peptide and a polypeptide of either herbicide tolerant protein, or δ-endotoxin Cry2A, or vegetative insecticidal protein VIP3A; (11) obtaining a second plant; and (iii) crossing the first and second plants to get a crossed transgenic progeny plant that has inherited the nucleic acid segments from the first plant.

The present invention precisely includes the progeny plant or seed from any of the transgenic plants, dicot or monocot, containing the whole or fewer polynucleotide sequences SEQ ID NO: 19.

### BEST METHOD OF CARRYING OUT THE INVENTION

Following processes in aggregation with the related sequence listings will further highlight the scope of present invention. The processes are described below:

### Cloning and Vector Construction

For the construction of the Agrobacterium-mediated transformation vector pFourB3G, typical restriction digestion and ligation techniques of polynucleotide fragments for gene cloning in subvectors were used.

The plasmid vector comprised the following cassettes: (i) a gene cassette containing a CaMV3SS promoter, the sequence from second intron of Petunia Cab gene for chlorophyll a/b binding protein gene, a chloroplast transit peptide (cTP) of Petunia Cab gene for chlorophyll a/b binding protein gene, a sequence encoding the cotton-optimized synthetic EPSPS gene conferring herbicide tolerance and a NOS polyadenylation sequence; (ii) second gene cassette comprising a CaMV35S promoter, the sequence from first intron of Brassica rapa Cab gene for chlorophyll a/b binding protein gene, a chloroplast transit peptide (cTP) of Ricin Cab gene for chlorophyll a/b binding protein gene, a sequence encoding the cotton-optimized VIP3A gene and a NOS polyadenylation sequence; (iii) the third gene cassette consisting of CaMV35S or GhSCFP promoter, sequence from first intron of G. hirsutum SCFP gene, a chloroplast transit peptide (cTP) of Petunia EPSPS gene, a sequence encoding the cotton-optimized Cry2Ab gene and a NOS polyadenylation sequence; (iv) and the fourth cassette comprising CaMV35S promoter, a sequence encoding protein conferring resistance to hygromycin or kanamycin and a NOS polyadenylation sequence.

The third gene cassette, alternatively, may also consist of PNZIP promoter and a sequence from first intron of PNZIP gene, in addition to above composition.

These gene cassettes were cloned within the T-DNA region of vector pFourB3G flanked by left and right border sequences. While the vector containing the above three cassettes and Cry2A under PNZIP promoter is given the name p4BG3.

Using standard Agrobacterium electroporation transformation technique, the above vectors were transformed into Agrobacterium tumefaciens strain LB4404, and the transformed cells were selected via kanamycin.

### Cotton Plant Transformation

The transgenic cotton plants were generated by standard Agrobacterium-mediated transformation method using germinating embryos of G. hirsutum cv FB202 and FB203 as optimized by Kiani et al. (2013).

For sterilization, FB202 and FB203 delinted seeds were surface sterilized for 60 seconds by continuously swirling the flask with 5% SDS and 5% Mercuric chloride using enough water to cover the seed. The seeds were subsequently washed in sterile water until no foam was seen. Finally, the sterilized seeds were soaked in 10 mL of autoclaved distilled water. The flask was covered with black cloth and seeds were allowed to germinate in dark at 30° C. for 36 hours before isolation of embryos. A 10 mL culture of Agrobacterium containing the pFourB3G and/or p4BG3 constructs was grown in YEP media broth separately overnight using suitable selection antibiotics. The culture was then centrifuged at 4° C. and the pellet was dissolved in 10 mL of MS broth, which was further diluted with MS broth (9; 1) in new sterile 50 mL Falcon tube. Germinating cotton embryos were taken out manually removing seed coat as well as cotyledonous tissues. Each isolated embryo was given a minor cut towards shoot-apex and placed in diluted Agrobacterium culture supplemented with Acetosyringone (Sigma-Aldrich™) and were allowed to co-cultivate for 1 hour in a shaker set at 30° C. Agrobacterium-treated embryos were then blotted on sterile filter paper to remove excess bacteria. The embryos were transferred to plates containing MS medium (MS salts, B5 vitamins, 2 mg/L NAA, 0.1 mg/L kinetin, 30 g/L sucrose, 4 g/L Phytogel, 500 mg/L cefotaxime sodium-salt, pH 5.8). No selection antibiotic hygromycin or kanamycin was added. The plates were wrapped with parafilm and incubated in the light at 28-30° C. for three days. The embryos grew in size and turned green. At fourth day, the healthy growing embryos from plates were shifted to 25×200 mm test tubes containing MS media (same composition as above) supplemented with appropriate selection antibiotics and were kept under 14 hours light and 10 hours dark conditions for three to four months until healthy shoots were formed. During this period, shoots were transferred to fresh MS selection media after every three weeks. Fully developed shoots were then shifted to rooting MS medium supplemented with hormones and containing no selection antibiotics. The shoots with healthy roots were then given the status of putative transgenic plants and were shifted to small pots containing soil, peat and bhall in specific ratio. These plants were then acclimatized.

### Identification and Selection of Transgenics

DNA was extracted from leaves and tested through standard polymerase chain reaction technique using gene specific primer sequences (SEQ ID NO: 1 to SEQ ID NO: 12) for the presence of transgenes (EPSPS, Cry2A and VIP3A) in putative acclimatized transgenic plants. The positive plant events were identified and screened through glyphosate spray assay as well as laboratory insect bioassays for their insecticidal activity against lepidopteran insects; that is, American bollworm (Helicoverpa armigera), Spotted bollworm (Earias insulana), Fall Armyworm (Spodoptera frugiperda) and Pink bollworm (Pictinophora gossypiella).

### Antibodies Production

Three male albino rabbits, approximately weighing 2Kg, were subcutaneously injected at multiple sites separately with purified EPSPS, Cry2A and VIP3A proteins. The rabbits were labeled accordingly and well fed and were injected with proteins further after fifteen days. The antibody titer was checked by ELISA taking 5 mL blood of each rabbit. Whole blood was isolated by cardiac puncture after two months. Serum was isolated by standard procedure and stored at −20° C. Pre-immune control serum was obtained from rabbits before immunization.

### Purification of Antibodies

Rabbit monoclonal anti-EPSPS, anti-Cry2A and anti-VIP3A antibodies purified on Protein-A affinity resin. Purified antibodies were dialyzed against PBS, dispensed in aliquots and stored frozen at −20° C. ELISA titer was again carried out to check activity of each purified antibody.

### Characterization of Antibodies

### Extraction of Protein

200 mg of transgenic as well as non-transgenic control plant material (leaves, stem, root etc.) was ground in liquid Nitrogen in pre-chilled sterile mortar. Fine dry ground powder was transferred to sterile 1.5 mL eppendorf tube and added 300 microL protein extraction buffer (0.5M EDTA, 0.5M Glycerol, 0.5M NaCl, 20 mM Tris-HCl pH 7.5, 20 mM NH4Cl, 0.5M PMSF, 10 mM DTT). After homogenization by vortexing, samples were incubated for one hour to overnight at 4° C. refrigerator and centrifuged for 15 minutes at 4° C. at maximum speed. Supernatent was taken in another eppendorf and using Bradford reagent, extracted protein was quantified on spectrophotometer. The samples were diluted by 1:10 for further analysis.

### Enzyme Linked Immunosorbant Assay (ELISA)

EPSPS, Cry2A and VIP3A proteins expressed in chloroplast as well as cytosol were detected by indirect ELISA. Before starting ELISA, plant protein samples and B. thuringiensis strains for periplasmic fractions were kept in boiling water for 10 minutes to denature the endogenous alkaline phosphatase or kinase enzyme activities. All denatured samples and purified protein were mixed with 50 mM carbonate buffer (pH 9.5) and dispensed into 96-well micro-titer plates accordingly and incubated at 37° C. overnight. Unbound antigen was rinsed out with Tris-buffer saline and Tween-20 (TBST). Blocking of unbound non-specific sites was done by 5% BSA/TBS Blocking Buffer and allowed to react with anti-EPSPS, anti-Cry2A and anti-VIP3A antibodies respectively. After standard washings, the bound antibodies were detected with AP-conjugated goat antirabbit IgG using BCIP/NBT substrate. ELISA reaction was stopped by adding 1N HCl. Absorbance rate was estimated at 430 nm spectrum, using negative control as blank.

A graph was plotted with standards between optical densities (OD) of different concentrations of standards. The concentrations of GTG, Cry2A and VIP3A were determined by placing their respective OD values on standard graph curve. Following formula was used for protein quantification:

$$Transgenic protein = Conc on graph \times \frac{[ 500 \times mg of tissue taken ]}{1000} \times dil ution factor ( Micro G \text{/} g leaf tissue )$$

### Immuno Dot Blot

Dot blot analysis was carried out for quick screening of extracted protein samples containing EPSPS, Cry2A and/or VIP3A expressed proteins in chloroplast and cytosol of transgenic cotton plants. Denatured Bt periplasmic fractions, purified protein, Triple-gene transformed and non-transformed control protein samples were applied (10 microL) onto nitrocellulose membrane. After drying, the unbound parts of the membrane were blocked in 5% BSA/TBS Blocking Buffer. The blot was washed three times with 1×PBS and added primary antibodies (anti-Cry1Ac, anti-GTG, anti-VIP3A Rabbit 1:5000) and incubated at 37° C. for one hour. The blot was given three washings with 1×PBS and incubated with secondary IgG (anti-IgG Rabbit mouse AP-conjugated). After one hour incubation with anti-IgG, the blot was again washed three times with 1×PBS and added BCIP/NBT substrate and incubated at 37° C. for 30 minutes for detection of transgenic proteins.

### Genomic DNA Extraction

For PCR analysis, genomic DNA was isolated from leaves of transgenic plants using protocol optimized by Kiani et al. (2013). Fresh leaf samples (300 mg) were plucked and immediately were kept in liquid Nitrogen container before grinding. Each sample was finely ground in pre-chilled sterile mortar & pestle using liquid Nitrogen. Dry powder was taken into fresh eppendorf and mixed thoroughly with pre-heated DNA extraction buffer (2% CTAB, 1% Mercaptoethanol, 2M NaCl, 200 mM EDTA and 100 mM Tris-HCl, RNase A). After incubation at 700 C for 30 minutes, added one volume of Phenol (pH 8), vortexed and spinned for 10 minutes at maximum speed. Supernatent was further extracted with equal volume of Chloroform: Isoamylalcohol (24:1) and spinned again. Supernatent was taken and added 0.7 volume of isopropanol and kept at room temperature for one hour. After spinning, the DNA pellet was washed twice with 70% freshly prepared ethanol, air dried and resuspended in 50 microL sterile water. DNA was quantified on 0.8% Agarose gel.

### Polymerase Chain Reaction (PCR)

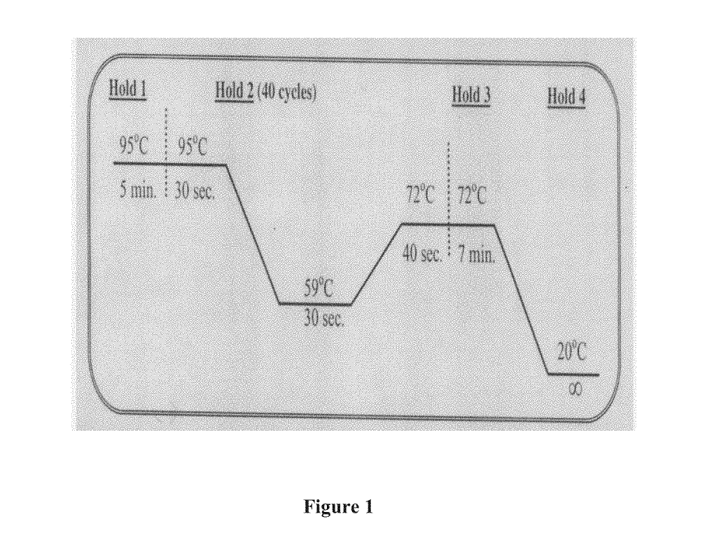

Referring to FIG. 1 illustrates a PCR amplification of GTG, Cry2A and VIP3A genes from genomic DNA was carried out using gene-specific primers (SEQ ID NOs: 1-12) in reaction volume of 25 microL. PCR mix was comprised as: template DNA 200 ng, gene specific primers forward & reverse 20 picoM each; dNTPs mix 3 mM, 1×Taq buffer, 2 units of Taq polymerase (Thermo Scientific). PCR reaction was carried out in Thermocycler (Applied Biosciences) with the conditions: 95° C. 5 min, (95° C. 30 sec, 59° C. 30 sec, 72° C. 40 sec)×40 cycles, 72° C. 7 min, 20° C. hold.

### Agarose Gel Electrophoresis

The PCR amplified gene fragments were run on 1% agarose gel containing Ethidium bromide (0.5 microG/mL) in 1% TAE buffer. PCR mix was with 4 microL bromo-phenol loading dye before loading in gel wells. Electrophoresis was carried out at 100V for 30 min in Gel Electrophoresis apparatus (BioRad) and was observed under UV in Gel Documentation apparatus (UVP, USA).

### BRIEF DESCRIPTION OF THE SEQUENCES

The sequence listing submitted form part of the present description and are given to further validate certain features of the present invention. Reference of these sequences may enhance the vision and scope of the present invention and specific embodiments described herein.

1. A plant or a part of plant conferring enhanced resistance to insect feeding on the plant or the part of the plant comprising:
a plurality of cassettes having a plurality of polynucleotide sequences assisting a triple gene for encoding insecticidal toxins and a herbicide tolerant protein;
each of the plurality of cassettes having each of the triple genes with a 5′ end attached to a promoter operably joint to an intron sequence, a cTP, and a 3′ end attached to a NOS terminator, and
wherein the triple gene is encoded so as to provide transgenic plants having chloroplast-targeted higher expression to yield fusion proteins i.e. insecticidal toxins and the herbicide tolerant proteins increasing effectiveness against the insect feeding on the plant by acting in synergist way and decreasing possibility of resistance against the insecticidal toxins and the herbicide tolerant protein.

2. The plant or the part of plant as claimed in claim 1, wherein the plurality of cassettes includes a first cassette, a second cassette, a third cassette, and a fourth cassette are within a T-DNA region of vector flanked by a left border sequence and a right border sequence.

3. The plant or the part of plant as claimed in claim 1, wherein the intron sequences and the cTP have a SEQ ID 13, SEQ ID 14, and SEQ ID 15 for targeted expression of protein coded from the triple gene.

4. The plant or the part of plant as claimed in claim 1, wherein the first cassette coding the herbicide tolerant EPSPS gene having a SEQ ID 17 operably linked to a 35CaMV35S promoter, a second intron of a Petunia Cab gene, and the cTP of the Petunia Cab gene at the 5′ terminal.

5. The plant or the part of plant as claimed in claim 1, wherein the second cassette coding the insecticidal vip3 gene having a SEQ ID 18 operably linked to a 35CaMV35S promoter, a first intron of Brassica rapa Cab gene, and the cTP of Ricin Cab gene at the 5′ terminal.

6. The plant or the part of plant as claim as claimed 1, wherein the third cassette coding the insecticidal Cry2ab gene having a SEQ ID 16 operably linked to a 35CaMV35S promoter, a first intron of G. hirusutum, and the cTP of the Petunia EPSPS gene at the 5′ terminal.

7. The plant or the part of plant as claimed 6, wherein the third cassette coding the insecticidal Cry2ab gene having a SEQ ID 16 may be operably linked to a GhSCFP promoter.

8. The plant or the part of plant as claimed in claim 6, wherein the third cassette coding the insecticidal Cry2ab gene having a SEQ ID 16 may be operably linked to PNZIP promoter and a first intron of pnzip gene.

9. The plant or the part of plant as claimed in claim 6, wherein the fourth cassette includes a 35CaMV35S promoter linked to a gene coding a protein conferring antibiotic resistance and a NOS adenylation sequence.

10. The plant or the part of plant as claimed in claim 9, wherein the antibiotic resistance is to hygromycin or kanamycin.

11. The plant or the part of plant as claimed in claim 1, wherein the plant is a monocotyledonous plant selected from the group consisting of maize, rice, wheat, and sugarcane plant.

12. The plant or the part of plant as claimed in claim 1, wherein the plant is a dicotyledonous plant selected from the group consisting of cotton, tomato, and potato plant.

13. The plant or the part of plant as claimed in claim 12, wherein the dicotyledonous plant is a cotton plant.

14. The plant or the part of plant as claimed in claim 1, wherein the yield of fusion protein is derived by a 4B3G transgenic event.

15. The plant or the part of plant as claimed in claim 1, wherein the yield of fusion protein is derived by a 4BG3 transgenic event.

16. The plant or the part of plant as claimed in claim 1, wherein the fusion protein having a SEQ ID 21, 22 and 23 are present in the plant or the part of plant.

17. A method to achieve expression of a triple gene for conferring enhanced resistance to insect feeding on the plant or the part of the plant, without undesirably affecting the normal phenotype and agronomic characteristics of a transgenic plant comprising the steps of:
designing and constructing a plurality of cassettes, wherein each of the plurality of cassettes has each gene of the triple genes having a 5′ end attached to a promoter operably joint to an intron sequence, a cTP, and a 3′ end attached to a NOS terminator are inserted within a T-DNA region of vector flanked by a left border sequence and a right border sequence;
transforming plants using an Agrobacterium mediated transformation vector having the T-DNA with the plurality of cassettes expressing the triple gene, so that the transgenic plant expresses the chloroplast-targeted higher expression of fusion proteins.

18. The method as claimed in claim 17, wherein the plurality of cassettes has a first cassette coding the herbicide tolerant EPSPS gene having a SEQ ID 17 operably linked to a 35CaMV35S promoter, a second intron of a Petunia Cab gene, and the cTP of the Petunia Cab gene at the 5′ terminal.

19. The method as claimed in claim 17, wherein the plurality of cassettes has a second cassette coding the insecticidal vip3 gene having a SEQ ID 18 operably linked to a 35CaMV35S promoter, a first intron of Brassica rapa Cab gene, and the cTP of Ricin Cab gene at the 5′ terminal.

20. The method as claimed in claim 17, wherein the plurality of cassettes has a third cassette coding the insecticidal Cry2ab gene having a SEQ ID 16 operably linked to a 35CaMV35S promoter, a first intron of G. hirusutum, and the cTP of the Petunia EPSPS gene at the 5′ terminal.

21. The method as claimed in claim 20, wherein the third cassette coding the insecticidal Cry2ab gene having a SEQ ID 16 may be operably linked to a GhSCFP promoter.

22. The method as claimed in claim 20, wherein the third cassette coding the insecticidal Cry2ab gene having a SEQ ID 16 may be operably linked to PNZIP promoter and a first intron of pnzip gene.

23. The method as claimed in claim 18 or claim 19 or claim 20, wherein the cTP is joint at the N-terminal for the enhanced targeted expression of Cry2A, VIP3A and GTG gene in transgenic cotton plants.

24. The method as claimed in claim 17, wherein, the plurality of cassettes includes a fourth cassette includes a 35CaMV35S promoter linked to a gene coding a protein conferring antibiotic resistance and a NOS adenylation sequence.

25. The method as claimed in claim 24, wherein the antibiotic resistance is to hygromycin or kanamycin.

26. The method as claimed in claim 17 or claim 18 or claim 19 or claim 20, wherein the intron sequences and the cTP have a SEQ ID 13, SEQ ID 14, and SEQ ID 15 for targeted expression of protein coded from the triple gene.

27. The method as claimed in claim 17, wherein the fusion protein having a SEQ ID 21, SEQ ID 22 and SEQ ID 23 are present in the plant or the part of plant.

28. A process for detection of a fusion protein i.e. insecticidal toxins and a herbicide tolerant protein coded by a triple gene conferring enhanced resistance to insect feeding on the plant or the part of the plant, in transgenic, the process consisting of steps of:
Producing of antibodies specific to purified EPSPS protein, Cry2Aprotein and VIP3A protein;
Purifying of antibodies using purified Protein A affinity resin;
Characterizing of antibodies using indirect ELISA; and
Extracting of protein.

29. The process of claim 28, wherein antibodies are detected through ELISA by the process consisting of:
denaturing the purified proteins to inactivate endogenous alkaline phosphatase;
loading denatured samples in microtiter plate;
blocking the unbound non-specific sites with BSA/TBS blocking buffer,
allowing the protein samples to react with antibodies such as anti-EPSPS, anti-Cry2A and anti-VIP3A respectively;
detecting bound antibodies such as anti-EPSPS, anti-Cry2A and anti-VIP3A with AP-conjugated goat antirabbit IgG using BCIP/NBT substrate, after standard washings;
stopping the ELISA reaction by adding 1N HCl and estimating the absorbance rate at 430 nm spectrum, using negative control as blank; and
plotting a graph with standards between optical densities (OD) of different concentrations of standards, determining the concentrations of GTG, Cry2A and VIP3A by placing their respective OD values on standard graph curve and using the following formula for protein quantification:
$$Transgenic protein = Conc on graph \times \frac{[ 500 \times mg of tissue taken ]}{1000} \times dil ution factor ( Micro G \text{/} g leaf tissue )$$

30. A process of DNA extraction from parts of transgenic plants and a PCR analysis, the process comprising steps of:
plucking fresh leaf samples and immediately keeping in liquid Nitrogen container;
grinding finely leaves or other parts of transgenic plants using in pre-chilled sterile mortar & pestle using liquid Nitrogen;
taking dry powder and mixing thoroughly with pre-heated DNA extraction buffer (2% CTAB, 1% Mercaptoethanol, 2M NaCl, 200 mM EDTA and 100 mM Tris-HCl, RNase A);
incubating at 700C for 30 minutes in mixture of dry powder and pre-heated DNA extraction buffer,
adding one volume of Phenol (pH 8), vortexing and spinning for 10 minutes at maximum speed and extracting supernatant with equal volume of Chloroform: Isoamylalcohol (24:1) and spinning again;
taking supernatent and adding 0.7 volume of isopropanol and kept at room temperature for one hour, after spinning, washing the DNA pellet twice with 70% freshly prepared ethanol, air dried and resuspending in 50 microL sterile water, and
quantifying DNA on 0.8% Agarose gel through electrophoresis.

31. The process of claim 30, wherein in PCR analysis the PCR amplification of EPSPS, Cry2A and VIP3A genes from genomic DNA is carried out using gene-specific primers having SEQ ID NOs: 1-12.


SEQUENCE LISTING (23 sequences)

SEQ ID NO: 1 - DNA, 23 bp, Artificial Sequence
  FOURB cotton nucleotide motif
atgagttccgttttgaactcagg

SEQ ID NO: 2 - DNA, 24 bp, Artificial Sequence
  FOURB cotton nucleotide motif
tcaatagagtggggagatattggt

SEQ ID NO: 3 - DNA, 21 bp, Artificial Sequence
  FOURB cotton nucleotide motif
atgcttcatggagcttcatca

SEQ ID NO: 4 - DNA, 23 bp, Artificial Sequence
  FOURB cotton nucleotide motif
ctatgctgcttttgtgtcagaga

SEQ ID NO: 5 - DNA, 29 bp, Artificial Sequence
  FOURB cotton nucleotide motif
atgaacaagaataatactaaacttagcac

SEQ ID NO: 6 - DNA, 23 bp, Artificial Sequence
  FOURB cotton nucleotide motif
atcctatttaatgctcacgtcgt

SEQ ID NO: 7 - DNA, 21 bp, Artificial Sequence
  FOURB cotton nucleotide motif
actccacctaccaagcattca

SEQ ID NO: 8 - DNA, 20 bp, Artificial Sequence
  FOURB cotton nucleotide motif
gccactgatgctgaaatcct

SEQ ID NO: 9 - DNA, 19 bp, Artificial Sequence
  cotton nucleotide motif
gcaaaggcctcactctcca

SEQ ID NO: 10 - DNA, 22 bp, Artificial Sequence
  FOURB cotton nucleotide motif
caccaccaaagctaccaatctt

SEQ ID NO: 11 - DNA, 23 bp, Artificial Sequence
  FOURB cotton nucleotide motif
ttctttctgtttgatgttgtagt

SEQ ID NO: 12 - DNA, 20 bp, Artificial Sequence
  FOURB cotton nucleotide motif
ggaggagtagaagatgccct

SEQ ID NO: 13 - DNA, 393 bp, Artificial Sequence
  FOURB cotton nucleotide motif
tatactttttaccactgcacccccccccaaagaccagtttttaactccacctaccaagcattcaagaagcaccaaccaacttaattaccagcttaacaagacagtacaggtttctgggatatttgtagtctctcaaggacatcaccacctccactcaccttcccatttttctctagcatggcacaaattaacaacatggctcaagggatacaaacccttaatcccaattccaatttccataaaccccaagttcctaaatcttcaagttttcttgtttttggatctaaaaaactgaaaaattcagcaaattctatgttggttttgaaaaaagattcaatttttatgcaaaagttttgttcctttaggatttcagcatcagtggctacagcacag

SEQ ID NO: 14 - DNA, 249 bp, Artificial Sequence
  FOURB cotton motif nucleotide
ctgggcaaaggcaaaggcctcactctccaaggaaaaatcagaaggaaatccaaatccaaatggactgaacctatcagcttctgcttaggtggtccatattgtgatcctcaaatccattatttttcatatatataccttcatatggcaacttctgcaattcaacaatcagcatttgctggacaaacagctcttaagtcacagaatgagcttgttaggaagattggtagctttggtggtggccgtgccacc

SEQ ID NO: 15 - DNA, 243 bp, Artificial Sequence
  FOURB cotton nucleotide motif
ggtcagacttttaaattctcagttttattgtttttctttctgtttgatgttgtagtaaagaaagatcttttattttttttcaattgtattataagtcatatggcaacacaagcacttgtgtcatcttctcttacttcttcagttgaaactgctagacaaattctaggtgctagatcttcctttgggtcttcaaggaagagcagctcctttgttgttagggcatcttctactcctcctgtcaag

SEQ ID NO: 16 - DNA, 1902 bp, Artificial Sequence
  FOURB cotton nucleotide motif
atgaattccgttttgaactcaggcagaactaccatctgcgatgcctacaatgttgctgcacacgacccgttttctttccagcataagagcctcgatactgtccaaaaggaatggaccgagtggaaaaaaaataatcacagtctttatctagatccaattgttggtacagtggcttcttttcttctgaagaaggttggaagtttagttgggaaacgaatattgtcagagttaagaaatcttattttcccgtctggatcaactaaccttatgcaagatatcctgagagaaactgagaaattcttgaatcagcgtttgaacactgacaccctcgctagagtaaacgcagaattgacaggccttcaagctaatgttgaagagttcaaccgacaagtcgataacttcttgaatcctaacaggaatgcagtgccactttcaattacttcatctgtaaacactatgcagcaactgtttttaaacaggttaccccagttccagatgcaaggctatcagctccttttacttccacttttcgctcaagctgctaatctccacttgtcttttattagagatgtgattctgaatgcagatgaatgggggatctccgccgcaactttgagaacatatcgtgattacttaaaaaactacactagagactattccaattactgtatcaacacatatcaatccgcttttaagggtctcaatactagacttcacgatatgctcgaattccgtacctatatgtttctcaatgtatttgagtatgtgtctatctggtctctttttaagtaccagtcacttcttgtctcctctggagcaaatctttatgcctctggttctgggcctcaacaaacccaaagctttacctctcaggattggcctttcttatacagcttatttcaagtcaactctaattatgtgctgaatggtttttccggtgcaagactttctaacactttccctaatattgtgggtttaccaggatccacaaccacacatgcacttttggctgctcgtgttaactattctggcggaattagcagcggagacattggtgccagtccgtttaatcaaaatttcaattgctctacttttcttcctccacttttgacaccatttgtaagatcatggctggactcaggctcagatagagagggtgttgccaccgttacaaactggcaaaccgaaagtttcgagactacccttggtctgaggtctggtgccttcactgcaagaggaaattcaaactactttcctgattatttcattaggaatattagcggtgttcccctggtggttaggaacgaggacttacgaaggccccttcattataatgaaatcaggaacatcgcttctcctagtggtactccggggggtgcccgagcatatatggttagcgttcacaacaggaaaaacaatattcatgctgttcatgaaaacgggtccatgattcatctggcccctaacgattacactggctttacaatcagtccgatccacgcaacacaagttaataatcaaactcgtacctttatctccgaaaagtttggtaaccaaggtgatagtcttcgattcgagcaaaacaacactactgctcgttacactctgaggggaaacggaaactcttacaatctctacttgagggtctcctccattggcaacagtactatcagagtaactattaacggtagggtgtacacagctactaatgtaaataccacgacaaacaacgatggtgttaatgacaacggcgctcgattttcagatatcaacatcggtaacgttgtggcttcctccaattctgatgttcctttagacatcaacgtcactttaaatagtgggacgcagtttgatctcatgaacataatgctggttcctaccaatatctccccactctattga

SEQ ID NO: 17 - DNA, 1368 bp, Artificial Sequence
  FOURB cotton nucleotide motif
atgcttcatggagcttcatcaaggcctgctactgctaggaagtctagcgggctcagtggcaccgtgagaattcctggcgataaatccatttcacacaggtccttcatgttcggtggtctggcttctggtgaaacgcgtattacaggattgctggagggagaagatgttatcaatactggtaaagctatgcaggctatgggtgcaagaattcgtaaagaaggagatacgtggattattgatggagttggcaacggtggtctcttagctcctgaagcacctttagatttcggtaatgccgctaccggatgcaggctgacaatgggacttgtgggagtatatgatttcgatagtacctttatcggggatgcctcacttacaaagaggcctatgggtagggttcttaatcctttaagagagatgggggtgcaagttaaatccgaggatggtgatcgtcttccggttactcttagaggccccaagacccctactcctattacctacagagttcctatggcttcagcacaggtcaaatccgccgttttgttggctggactcaatacccctggtatcaccaccgtcatcgaacccattatgacccgagatcataccgagaagatgttgcaggggtttggtgcaaatctgacggtcgaaaccgatgcagatggtgtgagaactattagactcgaaggtcgtgggaagcttaccggtcaagtgattgatgtgccgggggatccatcctctactgctttcccccttgttgcagcacttcttgtccccggctccgacgttactatacttaacgtgctcatgaatcctactagaactggcctcatcttgactctgcaggagatgggtgctgatattgaagtgattaaccccagacttgcaggtggagaggatgttgccgatctgagagtgagatcatctacattgaagggtgtgacggttcctgaagatagggccccttccatgatagatgaatacccaattttggcagtagcagctgctttcgccgaaggtgcaaccgtcatgaacggattggaggagctcagggtcaaagagtccgatagattgtctgccgttgcaaacgggttgaaattgaacggggtggactgtgacgagggcgaaacctctctggtcgtacgagggcgacctgacggtaagggtttgggaaacgcatctggcgctgccgtagctactcacctcgatcatcgaattgcaatgtcctttctggtgatgggattagtgagtgagaatcctgtgactgtggatgatgccaccatgattgctaccagttttcctgagttcatggatttaatggcaggtcttggagccaagattgaactctctgacacaaaagcagcatag

SEQ ID NO: 18 - DNA, 2373 bp, Artificial Sequence
  FOURB cotton nucleotide motif
atgaacaagaataatactaaacttagcactagagctcttccttctttcattgattattttaatgggatctatggatttgctacaggaattaaagatattatgaacatgattttcaagacagacacgggaggtgacttaaccttagatgagatattaaaaaaccaacaacttcttaacgacatctccggtaaacttgatggggttaatggaagtctcaacgatcttatagctcagggtaatcttaatacagaattgagtaaggagatattgaagatcgctaacgagcaaaatcaagttcttaacgacgttaacaataagctcgatgccatcaatactatgttgagggtgtatctgcccaagatcacctcaatgttatcagatgtcatgaaacagaattatgctctgagccttcagatcgaatatctgagtaagcaattgcaagaaatttctgataagctggatattatcaatgtgaacgtgctcatcaacagcacccttacagagatcacacctgcatatcaaagaattaaatatgtaaacgagaaatttgaagaattaacatttgccacagaaacatctagtaaggtaaaaaaggatggatcccctgccgatattttggatgagttaacagagcttaccgagttggccaagtctgtaactaaaaacgacgtcgacgggtttgaattttaccttaataccttccatgacgtcatggtaggaaacaatcttttcgggcgatcagcccttaagaccgcctctgagttgattactaaggagaatgtgaaaactagcggttcagaagttgggaatgtgtataattttttgatcgtgctcaccgctcttcaagcaaaggctttcctcaccttaaccacttgccgaaaattgcttggattagcagacatcgactatactagcataatgaatgaacatctcaacaaagagaaagaagagttcagagtaaatatattgccgactctcagcaacaccttcagcaatcctaactatgcaaaggttaaaggatctgatgaggacgcaaaaatgattgtggaggccaaacccggtcatgcactcattggctttgagattagtaacgactccatcactgtcctcaaagtgtacgaagcaaagttgaaacaaaattaccaggtggacaaagattcccttagtgaagttatctatggtgatatggataaattgttgtgccccgatcagtccgaacagatctattatactaacaatatagtattccctaatgagtatgtgattaccaaaattgattttactaagaaaatgaagactctgaggtatgaggtcaccgctaacttttacgattcatccactggtgagattgatctcaacaagaagaaagtggaatcatcagaagctgaatatagaactctttctgcaaacgacgacggtgtttatatgccgctcggggtaatttctgagactttcctgacccctattaatggctttggtcttcaggccgatgagaactctcgacttatcacccttacttgtaagtcctatcttagggaattacttttagctactgatctttctaacaaagaaaccaaattaatagttccaccctctgggttcatatctaatattgtagaaaacggtagcatcgaggaagacaatcttgagccgtggaaagctaataacaagaatgcttatgtggaccataccggaggtgttaatggtactaaagctctttatgttcacaaagatggtggtatctctcagttcatcggggataaattgaagcctaaaacagagtacgttatacagtatactgtgaagggaaagccttctattcatcttaaggacgaaaatacagggtatatccattatgaggataccaacaataacttagaggattaccaaaccattaataagagatttactacagggacagatcttaaaggcgtttatcttatccttaagtcacagaacggggatgaagcttggggggataacttcattatcctcgaaattagtccttctgagaagcttctctcccccgaattgataaacactaataattggacctctacagggagcacaaatattagcggcaacacccttacactttaccaagggggacgtggaattcttaaacagaatcttcaactcgacagcttttccacttatagggtctatttctctgtctcaggtgacgccaatgtgagaatcagaaatagtagagaggtcctctttgagaagaggtatatgagtggcgccaaagatgtttcagaaatgttcactacaaaatttgagaaagacaacttttacatcgagctgtctcagggtaataacctgtatggtgggccaattgtacatttctacgacgtgagcattaaataggat

SEQ ID NO: 19 - DNA, 12178 bp, Artificial Sequence
  FOURB cotton nucleotide motif
ggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaaggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattcagtactagctccctacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagccggtctcggtcagacttttaaattctcagttttattgtttttctttctgtttgatgttgtagtaaagaaagatcctttattttttttcaattgtattataagtcatcatatgatggcaacacaagcacttgtgtcatcttctcttacttcttcagttgaaactgctagacaaattctaggtgctaggtcttcctttgggtcttcaaggaagagcagctcctttgttgttagggcatcttctactcctcctgtcaagccatggatgaacaagaataatactaaacttagcactagagctcttccttctttcattgattattttaatgggatctatggatttgctacaggaattaaagatattatgaacatgattttcaagacagacacgggaggtgacttaaccttagatgagatattaaaaaaccaacaacttcttaacgacatctccggtaaacttgatggggttaatggaagtctcaacgatcttatagctcagggtaatcttaatacagaattgagtaaggagatattgaagatcgctaacgagcaaaatcaagttcttaacgacgttaacaataagctcgatgccatcaatactatgttgagggtgtatctgcccaagatcacctcaatgttatcagatgtcatgaaacagaattatgctctgagccttcagatcgaatatctgagtaagcaattgcaagaaatttctgataagctggatattatcaatgtgaacgtgctcatcaacagcacccttacagagatcacacctgcatatcaaagaattaaatatgtaaacgagaaatttgaagaattaacatttgccacagaaacatctagtaaggtaaaaaaggatggatctcctgccgatattttggatgagttaacagagcttaccgagttggccaagtctgtaactaaaaacgacgtcgatgggtttgaattttaccttaataccttccacgatgtgatggtaggaaacaatcttttcgggcgatcagcccttaagaccgcctctgagttgattactaaggagaatgtgaaaactagcggttcagaagttgggaatgtgtataattttttgatcgtgctcaccgctcttcaagcaaaggctttcctcaccttaaccacttgccgaaaattgcttggattagcagacatcgactatactagcataatgaatgaacatctcaacaaagagaaagaagagttcagagtaaatatattgccgactctcagcaacaccttcagcaatcctaactatgcaaaggttaaaggatctgatgaggacgcaaaaatgattgtggaggccaaacccggtcatgcactcattggctttgagattagtaacgactccatcactgtcctcaaagtgtacgaagcaaagttgaaacaaaattaccaggtggacaaagattcccttagtgaagttatctatggtgatatggataaattgttgtgccccgatcagtccgaacaggtctattatactaacaatatagtattccctaatgagtatgtgattaccaaaattgattttactaagaaaatgaagactctgaggtatgaggtcaccgctaacttttacgattcatccactggtgagattgatctcaacaagaagaaagtggaatcatcagaagctgaatatagaactctttctgcaaacgacgacggtgtttatatgccgctcggggtaatttctgagactttcctgacccctattaatggctttggtcttcaggccgatgagaactctcgacttatcacccttacttgtaagtcctatcttagggaattacttttagctactgatctttctaacaaagaaaccaaattaatagttccaccctctgggttcatatctaatattgtagaaaacggtagcatcgaggaagacaatcttgagccgtggaaagctaataacaagaatgcttatgtggaccataccggaggtgttaatggtactaaagctctttatgttcacaaagatggtggtatctctcagttcatcggggataaattgaagcctaaaacagagtacgttatacagtatactgtgaagggaaagccttctattcatcttaaggacgaaaatacagggtatatccattatgaggataccaacaataacttagaggattaccaaaccattaataagagatttactacagggaccgatcttaaaggcgtttatcttatccttaagtcacagaacggggatgaagttaggggggataacttcattatcctcgaaattagtccttctgagaaacttctctcccccgaattgataaacactaataattggacctctacagggagcacaaatattagcggcaacacccttacactttaccaagggggacgtggagttcttaaacagaatcttcaactcgacagcttttccacttatagggtctatttctctgtctcaggtgacgccaatgtgagaatcagaaatagtagagaggtcctctttgagaagaggtatatgagtggcgccaaagatgtttcagaaatgttcactacaaaatttgagaaagacaacttttacatcgagctgtctcagggtaataacctgtatggtgggccaattgtacatttctacgacgtgagcattaaataggattctagaatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcagtactgaattcgagctcggtacccggggatccctcgagagctccctacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagccatcgatctgggcaaaggcaaaggcctcactctccaaggaaaaatcagaaggaaatccaaatccaaatggactgaacctatcagcttctgcttaggtggtccatattgtgatcctcaaatccattatttttcatatatataccttcacgtcgacatggcaacttctgcaattcaacaatcagcatttgctggacaaacagctcttaagtcacagaatgagcttgttaggaagattggtagctttggtggtggccgtgccaccccatggatgcttcatggagcttcatcaaggcctgctactgctaggaagtctagcgggctcagtggcaccgtgagagttcctggcgataaatccatttcacacaggtccttcatgttcggtggtctggcttctggtgaaacgcgtattacaggattgctggagggagaagatgttatcaatactggtaaagctatgcaggctatgggtgcaagaatttgtaaagaaggagatacgtggattattgatggagttggcaacggtggtctgttagctcctgaagcacctttagatttcggtaatgccgctaccggatgcaggctgacaatgggacttgtgggagtatatgatttcgatagtacctttatcggggatgcctcacttacaaagaggcctatgggtagggttcttaatcctttaagagagatgggggtgcaagttaaatccgaggatggtgatcgtcttccggttactcttagaggccccaagacccctactcctattacctacagagttcctatggcttcagcacaggtcaaatccgccgttttgttggctggactcaatacccctggtatcaccaccgtcatcgaacccattatgacccgagatcataccgagaagatgttgcaggggtttggtgcaaatctgacggtcgaaaccgatgcagatggtgtgagaactattagactcgaaggtcgtgggaaacttaccggtcaagtgattgatgtgccgggggttccatcctctactgctttcccccttgttgcagcacttcttgtccccggctccgacgttactatacttaacgtgctcatgaatcctactagaactggcctcatcttgactctgcaggagatgggtgctgatattgaagtgattaaccccagacttgcaggtggagaggatgttgccgatctgagagtgagatcatctacattgaagggtgtgacggttcctgaagatagggccccttccatgatagatgaatacccaattttggcagtagcagctgctttcgccgaaggtgcaaccgtcatgaacggattggaggagctcagggtcaaagagtccgatagattgtctgccgttgcaaacgggttgaaattgaacggggtggactgtgacgagggcgaaacctctctggtcgtacgagggcgacctgacggtaagggtttgggaaacgcatctggcgctgccgtagctactcacctcgatcatcgaattgcaatgtcctttctggtgatgggattagtgagtgagaatcctgtgactgtggatgatgccaccatgattgctaccagttttcctgagttcatggatttaatggcaggtcttggagccaagattgaactctctgacacaaaagcagcatagggtaccatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcctcgagggatcctctagagtcgacctgcaggcatgcaagcttgcggccgcagctccctacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccacccctactccaaaaatgtcaaagatacagtctcagaagaccaaagggctattgagacttttcaacaaagggtaatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacagccagatcttatactttttaccactgcacccccccccaaagaccagtttttaactccacctaccaagcattcaagaagcaccaaccaacttaattaccagcttaacaagacagtacaggtttctgggatatttgtagtctctcaaggacatcaccacctccactcaccttcccatttttctctagccatatgatggcacaaattaacaacatggctcaagggatacaaacccttaatcccaattccaatttccataaaccccaagttcctaaatcttcaagttttcttgtttttggatctaaaaaactgaaaaattcagcaaattctatgttggttttgaaaaaagattcaatttttatgcaaaagttttgttcctttaggatttcagcatcagtggctacagcacagccatggatgagttccgttttgaactcaggcagaactaccatctgcgatgcctacaatgttgctgcacacgacccgttttctttccagcataagagcctcgatactgtccaaaaggaatggaccgagtggaaaaagaataatcacagtctttatctaggtccaattgttggtacagtggcttcttttcttctgaagaaggttggaagtttagttgggaaacgaatattgtcagagttaagaaatcttattttcccgtctggatcaactaaccttatgcaagatatcctgagagaaactgagaaattcttgaatcagcgtttgaacactgacaccctcgctagagtaaacgcagaattgacaggccttcaagctaatgttgaagagttcaaccgacaagtcgataacttcttgaatcctaacaggaatgcagtgccactttcaattacttcatctgtaaacactatgcagcaactgtttttaaacaggttaccccagttccagatgcaaggctatcagctccttttacttccacttttcgctcaagctgctaatctccacttgtcttttattagagatgtgattctgaatgcagatgaatgggggatctccgccgcaactttgagaacatatcgtgattacttaaaaaactacactagagactattccaattactgtatcaacacatatcaatccgcttttaagggtctgaatactagacttcacgatatgctcgaatttcgtacctatatgtttctcaatgtatttgagtatgtgtctatctggtctgtttttaagtaccagtcacttcttgtctcctctggagcaaatctttatgcctctggttctgggcctcaacaaacccaaagctgtacctctcaggattggcctttcttatacagcttatttcaagtcaactctaattatgtgctgaatggtttttccggtgcaagactttctaacactttccctaatattgtgggtttaccaggttcgacaaccacacatgcacttttggctgctcgtgttaactattctggcggaattagcagcggagacattggtgccagtccgtttaatcaaaatttcaattgctctacttttcttcctccacttttgacaccatttgtaagatcatggctggactcaggctcagatagagagggtgttgccaccgttacaaactggcaaaccgaaagtttcgagactacccttggtctgaggtctggtgccttcactgcaagaggaaattcaaactactttcctgattatttcattaggaatattagcggtgttcccctggtggttaggaacgaggacttacgaaggcccctgcactataacgaaatcaggaacatcgcttctcctagtggtactccggggggtgcccgagcatatatggttagcgttcacaacaggaaaaacaatattcatgctgttcatgaaaacgggtccatgattcatctggcccctaacgattacactggctttacaatcagtccgatccacgcaacacaagttaataatcaaactcgtacctttatctccgaaaagtttggtaaccaaggtgatagtcttcgattcgagcaaaacaacactactgctcgttacactctgaggggaaacggaaactcttacaatctctacttgagggtcttctccattggcaacagtacgatcagagtaactattaacggtagggtgtacacagctactaatgtaaataccacgacaaacaacgatggtgttaatgacaacggcgctcgattttcagatatcaacatcggtaacgttgtggcttcctccaattctgatgttcctttagacatcaacgtcactttaaatagtgggacgcagtttgatctcatgaacataatgctggttcctaccaatatctccccactctattgacccgggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggcgcggccgcaagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgttta

SEQ ID NO: 20 - DNA, 2124 bp, Artificial Sequence
  FOURB cotton polypeptide motif
atggcacaaattaacaacatggctcaagggatacaaacccttaatcccaattccaatttccataaaccccaagttcctaaatcttcaagttttcttgtttttggatctaaaaaactgaaaaattcagcaaattctatgttggttttgaaaaaagattcaatttttatgcaaaagttttgttcctttaggatttcagcatcagtggctacagcacagccatggatgagttccgttttgaactcaggcagaactaccatctgcgatgcctacaatgttgctgcacacgacccgttttctttccagcataagagcctcgatactgtccaaaaggaatggaccgagtggaaaaagaataatcacagtctttatctaggtccaattgttggtacagtggcttcttttcttctgaagaaggttggaagtttagttgggaaacgaatattgtcagagttaagaaatcttattttcccgtctggatcaactaaccttatgcaagatatcctgagagaaactgagaaattcttgaatcagcgtttgaacactgacaccctcgctagagtaaacgcagaattgacaggccttcaagctaatgttgaagagttcaaccgacaagtcgataacttcttgaatcctaacaggaatgcagtgccactttcaattacttcatctgtaaacactatgcagcaactgtttttaaacaggttaccccagttccagatgcaaggctatcagctccttttacttccacttttcgctcaagctgctaatctccacttgtcttttattagagatgtgattctgaatgcagatgaatgggggatctccgccgcaactttgagaacatatcgtgattacttaaaaaactacactagagactattccaattactgtatcaacacatatcaatccgcttttaagggtctgaatactagacttcacgatatgctcgaatttcgtacctatatgtttctcaatgtatttgagtatgtgtctatctggtctgtttttaagtaccagtcacttcttgtctcctctggagcaaatctttatgcctctggttctgggcctcaacaaacccaaagctgtacctctcaggattggcctttcttatacagcttatttcaagtcaactctaattatgtgctgaatggtttttccggtgcaagactttctaacactttccctaatattgtgggtttaccaggttcgacaaccacacatgcacttttggctgctcgtgttaactattctggcggaattagcagcggagacattggtgccagtccgtttaatcaaaatttcaattgctctacttttcttcctccacttttgacaccatttgtaagatcatggctggactcaggctcagatagagagggtgttgccaccgttacaaactggcaaaccgaaagtttcgagactacccttggtctgaggtctggtgccttcactgcaagaggaaattcaaactactttcctgattatttcattaggaatattagcggtgttcccctggtggttaggaacgaggacttacgaaggcccctgcactataacgaaatcaggaacatcgcttctcctagtggtactccggggggtgcccgagcatatatggttagcgttcacaacaggaaaaacaatattcatgctgttcatgaaaacgggtccatgattcatctggcccctaacgattacactggctttacaatcagtccgatccacgcaacacaagttaataatcaaactcgtacctttatctccgaaaagtttggtaaccaaggtgatagtcttcgattcgagcaaaacaacactactgctcgttacactctgaggggaaacggaaactcttacaatctctacttgagggtcttctccattggcaacagtacgatcagagtaactattaacggtagggtgtacacagctactaatgtaaataccacgacaaacaacgatggtgttaatgacaacggcgctcgattttcagatatcaacatcggtaacgttgtggcttcctccaattctgatgttcctttagacatcaacgtcactttaaatagtgggacgcagtttgatctcatgaacataatgctggttcctaccaatatctccccactctattga

SEQ ID NO: 21 - DNA, 1476 bp, Artificial Sequence
  FOURB cotton polypeptide motif
atggcaacttctgcaattcaacaatcagcatttgctggacaaacagctcttaagtcacagaatgagcttgttaggaagattggtagctttggtggtggccgtgccaccatgcttcatggagcttcatcaaggcctgctactgctaggaagtctagcgggctcagtggcaccgtgagaattcctggcgataaatccatttcacacaggtccttcatgttcggtggtctggcttctggtgaaacgcgtattacaggattgctggagggagaagatgttatcaatactggtaaagctatgcaggctatgggtgcaagaattcgtaaagaaggagatacgtggattattgatggagttggcaacggtggtctcttagctcctgaagcacctttagatttcggtaatgccgctaccggatgcaggctgacaatgggacttgtgggagtatatgatttcgatagtacctttatcggggatgcctcacttacaaagaggcctatgggtagggttcttaatcctttaagagagatgggggtgcaagttaaatccgaggatggtgatcgtcttccggttactcttagaggccccaagacccctactcctattacctacagagttcctatggcttcagcacaggtcaaatccgccgttttgttggctggactcaatacccctggtatcaccaccgtcatcgaacccattatgacccgagatcataccgagaagatgttgcaggggtttggtgcaaatctgacggtcgaaaccgatgcagatggtgtgagaactattagactcgaaggtcgtgggaagcttaccggtcaagtgattgatgtgccgggggatccatcctctactgctttcccccttgttgcagcacttcttgtccccggctccgacgttactatacttaacgtgctcatgaatcctactagaactggcctcatcttgactctgcaggagatgggtgctgatattgaagtgattaaccccagacttgcaggtggagaggatgttgccgatctgagagtgagatcatctacattgaagggtgtgacggttcctgaagatagggccccttccatgatagatgaatacccaattttggcagtagcagctgctttcgccgaaggtgcaaccgtcatgaacggattggaggagctcagggtcaaagagtccgatagattgtctgccgttgcaaacgggttgaaattgaacggggtggactgtgacgagggcgaaacctctctggtcgtacgagggcgacctgacggtaagggtttgggaaacgcatctggcgctgccgtagctactcacctcgatcatcgaattgcaatgtcctttctggtgatgggattagtgagtgagaatcctgtgactgtggatgatgccaccatgattgctaccagttttcctgagttcatggatttaatggcaggtcttggagccaagattgaactctctgacacaaaagcagcatag

SEQ ID NO: 22 - DNA, 2514 bp, Artificial Sequence
  FOURB cotton polypeptide motif
atggcaacacaagcacttgtgtcatcttctcttacttcttcagttgaaactgctagacaaattctaggtgctagatcttcctttgggtcttcaaggaagagcagctcctttgttgttagggcatcttctactcctcctgtcaagatgaacaagaataatactaaacttagcactagagctcttccttctttcattgattattttaatgggatctatggatttgctacaggaattaaagatattatgaacatgattttcaagacagacacgggaggtgacttaaccttagatgagatattaaaaaaccaacaacttcttaacgacatctccggtaaacttgatggggttaatggaagtctcaacgatcttatagctcagggtaatcttaatacagaattgagtaaggagatattgaagatcgctaacgagcaaaatcaagttcttaacgacgttaacaataagctcgatgccatcaatactatgttgagggtgtatctgcccaagatcacctcaatgttatcagatgtcatgaaacagaattatgctctgagccttcagatcgaatatctgagtaagcaattgcaagaaatttctgataagctggatattatcaatgtgaacgtgctcatcaacagcacccttacagagatcacacctgcatatcaaagaattaaatatgtaaacgagaaatttgaagaattaacatttgccacagaaacatctagtaaggtaaaaaaggatggatcccctgccgatattttggatgagttaacagagcttaccgagttggccaagtctgtaactaaaaacgacgtcgacgggtttgaattttaccttaataccttccatgacgtcatggtaggaaacaatcttttcgggcgatcagcccttaagaccgcctctgagttgattactaaggagaatgtgaaaactagcggttcagaagttgggaatgtgtataattttttgatcgtgctcaccgctcttcaagcaaaggctttcctcaccttaaccacttgccgaaaattgcttggattagcagacatcgactatactagcataatgaatgaacatctcaacaaagagaaagaagagttcagagtaaatatattgccgactctcagcaacaccttcagcaatcctaactatgcaaaggttaaaggatctgatgaggacgcaaaaatgattgtggaggccaaacccggtcatgcactcattggctttgagattagtaacgactccatcactgtcctcaaagtgtacgaagcaaagttgaaacaaaattaccaggtggacaaagattcccttagtgaagttatctatggtgatatggataaattgttgtgccccgatcagtccgaacagatctattatactaacaatatagtattccctaatgagtatgtgattaccaaaattgattttactaagaaaatgaagactctgaggtatgaggtcaccgctaacttttacgattcatccactggtgagattgatctcaacaagaagaaagtggaatcatcagaagctgaatatagaactctttctgcaaacgacgacggtgtttatatgccgctcggggtaatttctgagactttcctgacccctattaatggctttggtcttcaggccgatgagaactctcgacttatcacccttacttgtaagtcctatcttagggaattacttttagctactgatctttctaacaaagaaaccaaattaatagttccaccctctgggttcatatctaatattgtagaaaacggtagcatcgaggaagacaatcttgagccgtggaaagctaataacaagaatgcttatgtggaccataccggaggtgttaatggtactaaagctctttatgttcacaaagatggtggtatctctcagttcatcggggataaattgaagcctaaaacagagtacgttatacagtatactgtgaagggaaagccttctattcatcttaaggacgaaaatacagggtatatccattatgaggataccaacaataacttagaggattaccaaaccattaataagagatttactacagggacagatcttaaaggcgtttatcttatccttaagtcacagaacggggatgaagcttggggggataacttcattatcctcgaaattagtccttctgagaagcttctctcccccgaattgataaacactaataattggacctctacagggagcacaaatattagcggcaacacccttacactttaccaagggggacgtggaattcttaaacagaatcttcaactcgacagcttttccacttatagggtctatttctctgtctcaggtgacgccaatgtgagaatcagaaatagtagagaggtcctctttgagaagaggtatatgagtggcgccaaagatgtttcagaaatgttcactacaaaatttgagaaagacaacttttacatcgagctgtctcagggtaataacctgtatggtgggccaattgtacatttctacgacgtgagcattaaatag

SEQ ID NO: 23 - DNA, 1558 bp, Artificial Sequence
  FOURB cotton polynucleotide motif
acatggggatgaggcagggtatgagtgctttgctttttttttttggttaaaattttttcctccaggttgagatcctctctcagattcgaattgagtaattgctatgcattatacctacactcagcctttcgatgagttgtctcgggggttttgcagtttgatacatggattgatggagtattaagatgtttgtttatggagataaaagtgttgagcattcatatttgttggatttggctcattagtaatgtgaaggagaggacacaactttgttgaaaatacaacattgttatatatggcaccaaacaatttctagtatagggataaccttagtcaagttaattaggttgttgatttaataactataaggaatgatcaattgacctttatggtttgagccgagaaatactcttgaagatacaagatcaactacagaaaattacttgtgtcaggcaaaataactcttgaaatattattctattattgtacccccaattatttcattataaatcccattaattctcattattttatcagtattctaccatcaaacttgactctccattatttcaccataaatagccattgtccaccctcaaatagtcattaatataatctaccatgaatgtcttgccatgaatactatgtcctctaccactataaaaagactctacaaccaacaagggaggagaccaagctctatagctctacttcaagctactcaagttcgagaatagtatttctacaaattctatacactccacagactctatgaattattcctagcttgagtgcaacaacgtacatgcatctccacacttcaaaacatagtgaaattcaatttgattcatcttaaaaatgcatgtaatttttgtttttacttcaattttcacattaaaccccttgtttcttgtcctatacatatgactctagaactaacatgcgcaataagagaattatgtgggaaataaattgtaattccgtgaggaaagaataaaggtgaatgttacaaatttaagattgctgagctaaactgcatcagcattggtgttccttccatttactccacgattatcttatcagctttggattggtaaagaatctgcaactccagattctacccaagttaaagatactatactactactgaatattgtcactgtgcaatgctataaagatttttattatattgtgaagaagattaagtatagattgtgtaccaataacattgtgaaacgtaccatgaacaacatcagccacaaaatacacaaaatgaccatgtaatcaagctggcctgtcacagtatgccacgtgtcagaccaatatttaatcccatggatttctttggatgagatgatactccatcactttcatccaattatatatcctctccagcacccatagcttcacagtacactctacccagaaaaaaaaatggcagcagaaatggcattggtaaggcccatatcgaagttcggcgccaccgccaaatcgaattcccgcggccgccactccgcggctgagcggcaggcggaaactggcacccttaagcgtgaga